(19) United States
(12) Patent Application Publication
(10) Pub. No.: US 2007/0049807 A1
(43) Pub. Date: Mar. 1, 2007

(54) Method for reconstructing the distribution of fluorescent elements in a diffusing medium

(75) Inventors: Anabela Da Silva, Grenoble (FR); Jean-Marc Dinten, Lyon (FR)

Correspondence Address: OLIFF & BERRIDGE, PLC, P.O. BOX 19928, ALEXANDRIA, VA, 22320, US

(73) Assignee: COMMISSARIAT A L'ENERGIE ATOMIQUE, Paris (FR)

(21) Appl. No.: 11/511,323

(22) Filed: Aug. 29, 2006

(30) Foreign Application Priority Data
Aug. 29, 2005 (FR) 05 08836

(51) Int. Cl. A61B 5/00

(52) U.S. Cl. 600/312

(57) ABSTRACT

The method enables the distribution of fluorescent elements in a diffusing medium having substantially a finite cylindrical shape to be reconstructed. The method comprises formulation of a plurality of first energy transfer functions respectively representative of the energy transfer between the punctual excitation light source and the fluorescent elements and formulation of a plurality of second energy transfer functions respectively representative of the energy transfer between the fluorescent elements and the detector. The first transfer functions and the second transfer functions can thus be Green functions solving the diffusion equation and corresponding to a finite cylindrical volume, the Green functions being expressed as a function of the modified Bessel functions.

### BACKGROUND OF THE INVENTION

The invention relates to a method for reconstructing the distribution of fluorescent elements in a diffusing medium having substantially a finite cylindrical shape, comprising a step of formulating energy transfer functions in the medium between at least one punctual excitation light source and at least one detector.

### STATE OF THE ART

In optic imagery and in diffusive optic tomography, the examined object, or at least the examined zone, often presents a cylindrical shape. It may be constituted by a cylindrical tube in which a mouse for example, or a part of the human body, is placed.

Conventional diffusive optic tomography comprises reconstruction of absorption and/or diffusion contrasts, whereas fluorescence diffusive optic tomography comprises reconstruction of the fluorophore concentration and/or of the lifetime of fluorescent molecules. In the latter case, the distribution of fluorescent markers in a diffusing medium is sought to be determined from measurement data.

Two approaches are conventionally to be found for processing the measurement data. In a first approach, which is analytical, an infinite cylinder is considered in order to make a two-dimensional approximation, which does not enable satisfactory results to be obtained in the case where the cylinder is not infinite. In a second approach, which is numerical and three-dimensional, a cylinder of finite size is considered. In this case, the method used is for example the finite elements, finite differences or boundary integrals method. However, these methods consume considerable computing interval.

In the article “The theoretical basis for the determination of optical pathlengths in tissue: temporal and frequency analysis” by S. R. Arridge and al. (Phys. Med. Biol., 1992, Vol. 37, No 7, 1531-1560), light propagation in biological tissue confined in a finite cylinder is given by analogy with heat conduction in solids, without however taking fluorescence into account. S. R. Arridge's article makes reference to the publication “Conduction of heat in solids” by H. S. Carslaw and al. (1986, Oxford at the Clarendon Press), which describes a solution of the heat conduction equation in a finite cylinder by means of Green functions. The expression is limited to the surface points of the cylinder.

### OBJECT OF THE INVENTION

The object of the invention is to remedy these shortcomings and, in particular, to propose a method for reconstructing the distribution of fluorescent elements in a diffusive medium having substantially the shape of a finite cylinder, this method enabling an analytical and three-dimensional approach to be used.

According to the invention, this object is achieved by the fact that the formulation step comprises formulation of a plurality of first energy transfer functions respectively representative of energy transfer between the punctual excitation light source and the fluorescent elements and formulation of a plurality of second energy transfer functions representative of energy transfer between the fluorescent elements and the detector.

According to a development of the invention, the first transfer functions and the second transfer functions are Green functions solving the diffusion equation and corresponding to a finite cylindrical volume, the Green functions being expressed as a function of the modified Bessel functions.

### BRIEF DESCRIPTION OF THE DRAWINGS

Other advantages and features will become more clearly apparent from the following description of particular embodiments of the invention given as non-restrictive examples only and represented in the accompanying drawings, in which:

FIG. 1 illustrates light propagation in a diffusing medium having substantially a finite cylindrical shape.

FIG. 2 represents the flowchart of a particular embodiment of the method according to the invention.

### DESCRIPTION OF PARTICULAR EMBODIMENTS

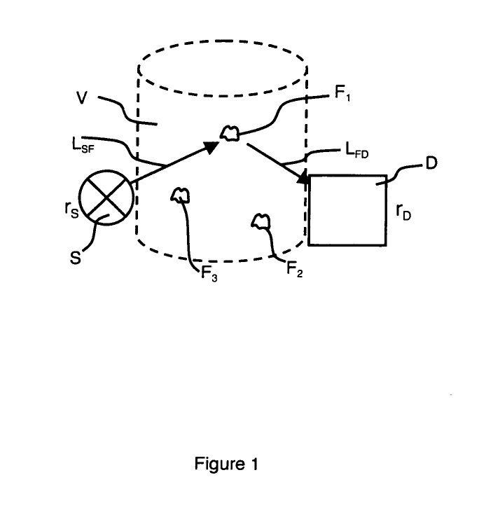

In FIG. 1, a punctual excitation light source S is placed at a point r_{S} and emits a light having a first wavelength λ_{SF} and an amplitude Q. The light emitted propagates in a volume V of a diffusing medium having substantially a finite cylindrical shape, wherein fluorescent elements F are arranged with a distribution that is sought to be determined. In FIG. 1, a first diffusive wave L_{SF} emitted by the source S excites a fluorescent element F₁ which then emits a radiation having a second wavelength λ_{FD}: the intensity of this second diffusive wave L_{FD} is measured by the detector D. The distribution of the fluorescent elements then has to be determined from the measurements made by the detector D. These measurements are performed from a set of source positions. In the case of several detectors, the measurements made by all the detectors will be taken into account.

In FIG. 1, two other fluorescent elements F₂ and F₃ are represented. The fluorescent elements F₁, F₂ and F₃ being located in the volume V, the detector receives a measured photon density φ^{M} composed of all the second waves L_{FD} emitted by all of the fluorescent elements F₁, F₂ and F₃. For the sake of clarity, the waves corresponding to the fluorescent elements F₂ and F₃ are not represented.

A first energy transfer function G(λ_{SF}, \vec{r}_{S}, \vec{r}_{F}) representative of the energy transfer between the punctual excitation light source S and the fluorescent elements F is defined, as is a second energy transfer function G(λ_{FD}, \vec{r}_{F}, \vec{r}_{D}) representative of the energy transfer between the fluorescent elements F and the detector D.

It can be shown that the theoretical photon density φ^{M} measured by the detector D can be expressed approximately by an integral comprising the product of the first and second transfer functions G:

$${ϕ}^{M} ( {{r}^{->}}_{S} , {{r}^{->}}_{D} ) = Q ( {r}_{S} ) {\int }_{v} G ( {λ}_{SF} , {{r}^{->}}_{S} , {{r}^{->}}_{F} ) β ( {{r}^{->}}_{F} ) G ( {λ}_{FD} , {{r}^{->}}_{F} , {{r}^{->}}_{D} ) ⅆ {{r}^{->}}_{F} , \qquad ( 1 )$$

where the parameter

$$β ( {{r}^{->}}_{F} ) = \frac{δμ ( {{r}^{->}}_{F} ) η}{1 - ⅈωτ ( {{r}^{->}}_{F} )} \qquad ( 2 )$$

depends on the quantum efficiency of the fluorescent element, the local absorption δμ(\vec{r}_{F}) due to the fluorescent elements and the damping (1−iωτ(\vec{r}_{F}))⁻¹ linked to the lifetime τ of the fluorescent elements F and to the pulse ω imposed externally to the radiation, the light being emitted in pulsed manner.

The first and second transfer functions G can be treated as Green functions determined by solving diffusion equations for the finite cylindrical volume V and for a given wavelength λ:
∇²G(λ,\vec{r},\vec{r}_{O})+k_{λ}G(λ,\vec{r},\vec{r}_{O})=δ(\vec{r}−\vec{r}_{O}) (3),
δ being the Dirac function and

$${k}_{λ}^{2} = - \frac{{μ}_{λ}}{{D}_{λ}} + \frac{ⅈω}{{c}_{n} {D}_{λ}}$$

the wave number. In these expressions, Cₙ, is the speed of the light in the medium, μ_{λ} is the absorption coefficient of the medium, D_{λ} is the diffusion coefficient and \vec{r} and \vec{r}_{O} are the spatial variables of the Green function. The parameters μ_{λ} and D_{λ} are evaluated at the corresponding wavelengths λ_{SF} and λ_{FD}. Moreover, the solutions of the diffusion equations must comply with boundary conditions on the surface delineating the cylindrical volume, for example Dirichlet conditions or Neumann conditions. The mathematical problem is thus analogous to the heat conduction problem in a condensed medium presenting a finite cylindrical shape.

For a finite cylindrical volume, the Green functions solving the diffusion equations and complying with the boundary conditions can be expressed as a function of the modified Bessel functions:

$$G ( λ , {r}^{->} , {{r}^{->}}_{O} ) = \frac{1}{{D}_{λ} π l} {\sum }_{p = 1}^{+ ∞} sin \frac{p π z}{l} sin \frac{p π {z}_{O}}{l} {\sum }_{n = - ∞}^{+ ∞} \frac{{I}_{n} ( {rk}_{p} ) {F}_{n} ( {ak}_{p} , {r}_{O} {k}_{p} )}{{I}_{n} ( {ak}_{p} )} cos n ( θ - {θ}_{O} ) \\ if \\ r \leq {r}_{O} , \\ G ( λ , {r}^{->} , {{r}^{->}}_{O} ) = \frac{1}{{D}_{λ} π l} {\sum }_{p = 1}^{+ ∞} sin \frac{p π z}{l} sin \frac{p π {z}_{O}}{l} {\sum }_{n = - ∞}^{+ ∞} \frac{{I}_{n} ( {r}_{O} {k}_{p} ) {F}_{n} ( {ak}_{p} , {rk}_{p} )}{{I}_{n} ( {ak}_{p} )} cos n ( θ - {θ}_{O} ) \\ if \\ r > {r}_{O} . \qquad ( 4 )$$

In these equations (4), l is the height of the cylinder, a is the radius of the cylinder, \vec{r}=(r,θ,z) and \vec{r}_{O}=(\vec{r}_{O},θ_{O},z_{O}) are the spatial variables of the Green function in cylindrical coordinates, and Fₙ(u,v)=Iₙ(u)Kₙ(v)−Kₙ(u)Iₙ(v), Iₙ and Kₙ are the modified Bessel functions respectively of first and second order type n. In addition, kₚ is defined kₚ=\sqrt{k_{λ}²+p²π²/l²}.

The equations (4) are then entered into the equation (1). For a discrete computation, the space of the cylindrical volume V is divided into N elementary volumes with a suitable meshing. The integral of the equation (1) is then replaced by a sum on the N elementary volumes dvⱼ constituting the cylindrical volume V:

$${ϕ}^{M} ( {{r}^{->}}_{S} , {{r}^{->}}_{D} ) = Q ( {{r}^{->}}_{S} ) {\sum }_{j = 1}^{N} G ( {λ}_{SF} , {{r}^{->}}_{S} , {{r}^{->}}_{j} ) β ( {{r}^{->}}_{j} ) F ( {λ}_{FD} , {{r}^{->}}_{j} , {{r}^{->}}_{D} ) {dv}_{j} . \qquad ( 5 )$$

According to the invention, the reconstruction method comprises formulation of a plurality of N first energy transfer functions G(λ_{SF},\vec{r}_{S},\vec{r}ⱼ) respectively representative of the energy transfer between the punctual excitation light source S and the fluorescent elements F and formulation of a plurality of N second energy transfer functions G(λ_{FD},\vec{r}ⱼ,\vec{r}_{D}) respectively representative of the energy transfer between the fluorescent elements and the detector. In this way, a first energy transfer function G(λ_{SF},\vec{r}_{S},\vec{r}ⱼ) and a second energy transfer function G(λ_{FD},\vec{r}ⱼ,\vec{r}_{D}) are associated with each elementary volume dvⱼ.

When a plurality of N_{S} punctual excitation light sources S and a plurality of N_{D} detectors D are considered, a matrix representation can be chosen:
[φ^{M}]_{N}_{_{D}}_{×N}_{_{S}}=[G_{FD}]_{N}_{_{D}}_{×N}[βdv]_{N×N}[G_{SF}]_{N×N}_{_{S}}[Q]_{Ndi S} (6).

In this equation, each column of [φ^{M}]_{N}_{_{D}}_{×N}_{_{S}} represents measurement on N_{D} detectors for a given source S.

For each source S and for each detector D, a conversion matrix J can be computed enabling the photon density φ^{M} measured at the parameter β to be linked linearly:

$${ϕ}_{SD}^{M} = {\sum }_{j = 1}^{N} {J}_{SDj} {β}_{j} . \qquad ( 7 )$$

The set of source-detector combinations enables the matrix equation to be constructed, which equation is then solved in a reconstruction algorithm either by calculating the error between the experimental measurements and this theoretical matrix equation (for example using the ART (Algebraic Reconstruction Technique) type error or algorithm back projection method) or by directly inverting the matrix J (for example by means of SVD (Single Value Decomposition) algorithms).

In the case of calculating the error between the experimental measurement and the theory, a theoretical distribution of the fluorescent elements adjusted in the course of calculation is used to reduce this error.

In the case of matrix inversion, the parameter β which depends on the distribution of the fluorescent elements is obtained in a first step by means of the local absorption δμ(\vec{r}_{F}) due to the fluorescent elements and by means of the damping (1−iωτ(\vec{r}_{F}))⁻¹ linked to the lifetime τ of the fluorescent elements F. Knowing the parameter β thus makes it possible to determine the distribution and the local concentration of the fluorescent elements.

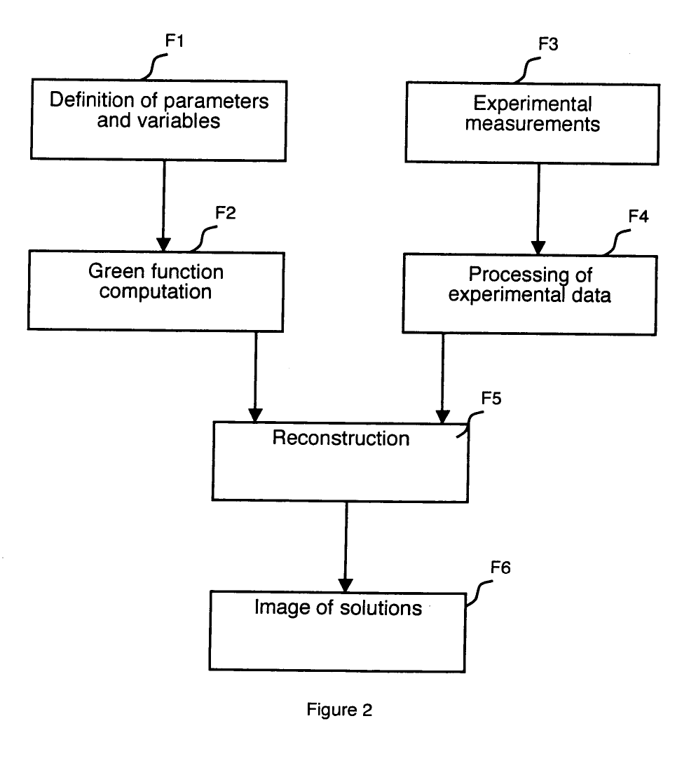

As represented in FIG. 2, the parameters and variables are defined (function F1 in FIG. 2) at the beginning of the reconstruction process. The geometry of the cylinder (height l and radius a), the positions of the sources (\vec{r}_{S}) and detectors (\vec{r}_{D}), the meshing of the medium, and the parameters constituting the medium such as the diffusion coefficient (D_{λ}), the absorption coefficient (μ_{λ}) and the wavelengths (λ_{SF}, λ_{FD}) are thus defined. Then the Green functions G are determined (function F2 in FIG. 2) according to the equations (4). The conversion matrix J can then be determined.

Furthermore, experimental measurement (function F3 in FIG. 2) and processing of the experimental data (function F4 in FIG. 2) enable the photon densities φ^{M} measured by the different detectors D to be determined. The parameters linked to the fluorescent elements F are then reconstructed (function F5 in FIG. 2). Additional processing (function F6 in FIG. 2) then enables the distribution of the fluorescent elements F and their concentration to be represented in image form.

The computing time required for computation of the Green functions according to the equations (4) can be optimized using a synthetic representation of the Green functions:

$$G = {\sum }_{p} {a}_{p} {u}_{p} \\ where \\ {a}_{p} = sin \frac{p π z}{l} sin \frac{p π {z}_{O}}{l} , \\ {u}_{p} = {\sum }_{n = - ∞}^{+ ∞} {g}_{n , p} \\ and \\ {g}_{n , p} = \frac{{I}_{n} ( {rk}_{p} ) {F}_{n} ( {ak}_{p} , {r}_{O} {k}_{p} )}{{I}_{n} ( {ak}_{p} )} cos n ( θ - {θ}_{O} ) . \qquad ( 8 )$$

It is more judicious to first calculate the functions aₚ (depending only on z) and uₚ (depending only on r and θ) separately, once and for all, and to then calculate their scalar product. The choice of the truncation order for the indices n and p can be made according to Shannon's theorem. For n, 100 is a sufficient order and for p, 20 is a sufficient order.

1. Method for reconstructing the distribution of fluorescent elements in a diffusing medium having substantially a finite cylindrical shape, comprising a step of formulating energy transfer functions in the medium between at least one punctual excitation light source and at least one detector, wherein the formulation step comprises:
formulation of a plurality of first energy transfer functions respectively representative of an energy transfer between the punctual excitation light source and the fluorescent elements
and formulation of a plurality of second energy transfer functions respectively representative of an energy transfer between the fluorescent elements and the detector.

2. Method according to claim 1, wherein the first transfer functions and the second transfer functions are Green functions solving a diffusion equation and corresponding to a finite cylindrical volume, the Green functions being expressed as a function of modified Bessel functions.
